(19) United States
(12) Patent Application Publication
(10) Pub. No.: US 2026/0146012 A1
(43) Pub. Date: May 28, 2026

(54) PROCESSES AND CATALYSTS FOR LINEAR ALPHA-OLEFIN PURIFICATION

(71) Applicant: SABIC GLOBAL TECHNOLOGIES B.V., Bergen op Zoom (NL)

(72) Inventors: Ashim Kumar Ghosh, Sugar Land, TX (US); Debashis Chakraborty, Houston, TX (US); Bander Bawareth, Jeddah (SA); Jonathan Banke, Sugar Land, TX (US)

(21) Appl. No.: 19/100,258

(22) PCT Filed: Jul. 13, 2023
(86) PCT No.: PCT/EP2023/069450

§ 371 Date: Jan. 31, 2025

(30) Foreign Application Priority Data
Aug. 2, 2022 (EP) 22188283.0

(51) Int. Cl. C07C 5/25; B01J 29/08; B01J 29/40; B01J 29/70; B01J 29/80; B01J 29/90

(52) U.S. Cl. CPC C07C 5/2518; B01J 29/082; B01J 29/084; B01J 29/40; B01J 29/7007; B01J 29/80; B01J 29/90; B01J 2229/42; C07C 2529/08

(57) ABSTRACT

The present disclosure provides for processes and catalysts for enhancing a linear alpha-olefin product stream from an oligomerization reaction. A catalyst composition including a first zeolite and a second zeolite is provided, the first and second zeolites each having an average pore size of greater than about 6 Å, wherein the catalyst composition has a Brönsted Acid Site (BAS) equivalent of about 1.0 mmol/g catalyst or less, and wherein the first zeolite is at least partially metal ion-exchanged and the second zeolite is at least partially in H+ form. A process for producing a purified linear-alpha olefin (LAO) is also provided, which includes introducing a LAO product stream comprising an alkyl-butene fraction into a catalyst bed comprising a zeolite catalyst and in fluid connection with the LAO product stream to catalytically isomerize the alkyl-butene fraction into an alkyl-pentene fraction.

### TECHNOLOGICAL FIELD

The present disclosure relates to processes and catalysts for enhancing the purity of a linear alpha-olefin product stream.

### BACKGROUND

Linear alpha-olefins (LAOs) represent a class of hydrocarbon compounds that are commercially utilized as raw materials in the petrochemical industry. Among LAOs, an important subclass of these compounds include unbranched olefins, whose double bond is located at a terminus of the chain, of which one of the most commercially-desirable compounds is 1-hexene. An essential component in many polymeric resins, including high-performance polyethylene (PE) compositions, 1-hexene is also valuable in many synthetic processes directed to the production of dyes, flavorants, and perfumes.

It is desirable to enhance the concentrations of 1-hexene and additional, commercially-valuable alpha-olefin products to maximal level, for instance, to concentration levels of around 99.5 wt. % or greater. However, such enhanced degrees of concentration and associated purity can be challenging using conventional separation processes due, for instance, the presence of one or more impurities characterized by boiling points that are comparable to the boiling point(s) of the alpha-olefin products of interest. Accordingly, there remains a need in the art for improvements in the enhancement and purification commercially-valuable LAO products.

### BRIEF SUMMARY

Example implementations of the present disclosure are directed to processes and catalytic articles for the purification and quantitative enhancement of a linear-alpha olefin (LAO) product stream. In particular, the processes and catalytic articles of the present disclosure involve the use of a zeolite catalyst positioned to receive a LAO product stream comprising an alkyl-butene fraction through one or more catalyst beds, the zeolite catalyst, may be regenerable under non-oxidative environment, and is capable of isomerizing the alkyl-butene fraction into an alkyl-pentene fraction. In this manner, the zeolite catalyst can enhance the purification of commercially-desirable LAOs to significantly greater concentrations.

The present disclosure includes, without limitation, the following embodiments.

Embodiment 1: A catalyst composition comprising a first zeolite and a second zeolite, the first and second zeolites each having an average pore size of greater than about 6 Å, wherein the catalyst composition comprises a Brönsted Acid Site (BAS) equivalent of about 1.0 mmol/g catalyst or less, and wherein the first zeolite is metal ion-exchanged and the second zeolite is in H⁺ form.

Embodiment 2: The catalyst composition of Embodiment 1, wherein the first zeolite is metal ion-exchanged with one or more metals selected from the group consisting of alkali metals, alkaline earth metals, and combinations thereof.

Embodiment 3: The catalyst composition of Embodiment 1 or 2, wherein the first zeolite is metal ion-exchanged with sodium.

Embodiment 4: The catalyst composition of any one of Embodiments 1-3, wherein each of the first and second zeolite has a 12-membered ring framework structure independently selected from the group consisting of AFI, AFR, AFS, AFY, ASV, ATO, ATS, BEA, BEC, BOG, BPH, BSV, CAN, CON, CZP, DFO, EMT, EON, EZT, FAU, GME, GON, IFR, ISV, ITG, IWR, IWS, IWV, IWW, JSR, LTF, LTL, MAZ, MEI, MOR, MOZ, MSE, MTW, NPO, OFF, OKO, OSI, RON, RWY, SAF, SAO, SBE, SBS, SBT, SEW, SFE, SFO, SFS, SFV, SOF, SOS, STO, SSF, SSY, USI, UWY, VET, and combinations thereof.

Embodiment 5: The catalyst composition of any one of Embodiments 1-4, wherein each of the first and second zeolite has a FAU or BEA framework structure, each of the first and second zeolite having a silica-to-alumina ratio (SAR) from about 1 to about 100, such as about 2 to about 30 or about 3 to about 20 or about 4 to about 15.

Embodiment 6: The catalyst composition of any one of Embodiments 1-5, wherein each of the first and second zeolite is zeolite X, zeolite Y, or beta zeolite.

Embodiment 7: The catalyst composition of any one of Embodiments 1-6, wherein the first zeolite is Na⁺ form zeolite Y (NaY) and the second zeolite is H⁺ form zeolite Y (HY).

Embodiment 8: The catalyst composition of any one of Embodiments 1-7, wherein the first and second zeolite are present in a weight ratio of first zeolite:second zeolite in a range of about 5:1 to about 15:1.

Embodiment 9: The catalyst composition of any one of Embodiments 1-8, wherein the wherein the catalyst composition comprises a Brönsted Acid Site (BAS) equivalent of about 0.9 mmol/g catalyst or less.

Embodiment 10: The catalyst composition of any one of Embodiments 1-9, wherein the wherein the catalyst composition comprises a Brönsted Acid Site (BAS) equivalent of about 0.5 mmol/g catalyst or less.

Embodiment 11: The catalyst composition of any one of Embodiments 1-10, further comprising a metal oxide binder.

Embodiment 12: Use of the catalyst composition of any one of Embodiments 1-11 to isomerize an alkyl-butene fraction into an alkyl-pentene fraction in a linear-alpha olefin (LAO) product stream.

Embodiment 13: A method of isomerizing an alkyl-butene fraction into an alkyl-pentene fraction in a linear-alpha olefin (LAO) product stream comprising contacting the LAO product stream with a catalyst composition of any one of Embodiments 1-11.

Embodiment 14: A process for producing a purified linear-alpha olefin (LAO) product stream, the process comprising: introducing a LAO product stream comprising an alkyl-butene fraction into a catalyst bed comprising a regenerable zeolite catalyst and in fluid connection with the LAO product stream to catalytically isomerize the alkyl-butene fraction into an alkyl-pentene fraction and form a treated LAO product stream; and directing the treated LAO product stream into at least one distillation column having a plurality of stacked stages to produce a purified LAO product stream.

Embodiment 15: The process of Embodiment 14, wherein the zeolite catalyst comprises a catalyst composition comprising a first zeolite and a second zeolite, the first and second zeolites each having an average pore size of greater than about 6 Å, wherein the catalyst composition comprises a Brönsted Acid Site (BAS) equivalent of about 1.0 mmol/g catalyst or less, and wherein the first zeolite is metal ion-exchanged and the second zeolite is in H⁺ form.

Embodiment 16: The process of Embodiment 14 or 15, wherein the alkyl-butene is 2-ethyl-1-butene (2E1B) and the alkyl-pentene is cis- or trans-3-methy-2-pentene.

Embodiment 17: The process of any one of Embodiments 14-16, wherein the LAO product stream has a 2-ethyl-1-butene (2E1B) concentration of about 0.7 wt. % or higher and the treated LAO product stream is characterized by a 2E1B concentration of equal to or less than about 0.3 wt. %.

Embodiment 18: The process of any one of Embodiments 14-17, wherein treatment with the zeolite catalyst catalytically isomerizes about 90% by weight or more of the 2-ethyl-1-butene (2E1B) and about 1.0% by weight or less of 1-hexene in the LAO product stream.

Embodiment 19: The process of any one of Embodiments 14-18, wherein the LAO product stream comprises 1-hexene in an amount of 90 wt. % or greater.

Embodiment 20: The process of any one of Embodiments 14-19, wherein the purified LAO product stream is characterized by a 1-hexene product purity of about 99.5 wt. % or greater.

Embodiment 21: The process of any one of Embodiments 14-20, wherein the introducing step occurs at one or more of the following conditions: a liquid hourly space velocity (LHSV) of about 0.5 h⁻¹ to about 10 h⁻¹; a pressure of about 5 pounds per square in gauge (psig) or less; and a temperature of about 40° C. to about 60° C.

Embodiment 22: The process of any one of Embodiments 14-21, wherein at least two catalyst beds comprising the zeolite catalyst are operated in parallel and in fluid connection with the LAO product stream such that at least one catalyst bed can be regenerated while continuing to treat the LAO product stream.

Embodiment 23: The process of any one of Embodiments 14-22, further comprising regenerating the catalyst bed by treating the catalyst bed in an environment substantially free of oxygen with an inert gas stream at a temperature in a range of about 150° C. to about 400° C.

These and other features, aspects, and advantages of the present disclosure will be apparent from a reading of the following detailed description in combination with the accompanying figures, which are briefly described below. The present disclosure includes any combination of two, three, four or more features or elements set forth in this disclosure, regardless of whether such features or elements are expressly combined or otherwise recited in a specific example implementation described herein. This disclosure is intended to be read holistically such that any separable features or elements of the disclosure, in any of its aspects and example implementations, should be viewed as combinable, unless the context of the disclosure clearly dictates or suggests otherwise.

It will be appreciated that the foregoing Brief Summary is provided for the general purposes of summarizing certain example implementations to provide a basic understanding of some aspects of the disclosure. Accordingly, it will be appreciated that the above-described example implementations are merely non-limiting examples that should not be interpreted as narrowing the scope or spirit of the disclosure in any way. Additional example implementations, aspects and advantages will become apparent from the following detailed description taken in conjunction with the accompanying figures which illustrate, by way of example, principles of some described example implementations.

### BRIEF DESCRIPTION OF THE FIGURES

Having thus described aspects of the disclosure in the foregoing general terms, reference will now be made to the accompanying drawings, which are not necessarily drawn to scale. The drawings are exemplary only, and should not be construed as limiting the disclosure.

FIG. 1 is a block diagram of an LAO product stream treatment system according to an example implementation of the present disclosure;

FIG. 2 graphically illustrates results of a catalytic isomerization of 2-ethyl-1-butene to 3-methyl 2-pentene (upper traces) as well as loss of 1-hexene (lower traces) in accordance with aspects of the present disclosure;

FIG. 3 graphically illustrates additional results of a catalytic isomerization of 2-ethyl-1-butene to 3-methyl 2-pentene (upper traces) as well as loss of 1-hexene (lower traces) at time points prior to and following catalyst regeneration in accordance with aspects of the present disclosure;

FIG. 4 graphically illustrates additional results of a catalytic isomerization of 2-ethyl-1-butene to 3-methyl 2-pentene (upper traces) as well as loss of 1-hexene (lower traces) at time points prior to and following catalyst regeneration in accordance with aspects of the present disclosure;

FIG. 5 graphically illustrates results of a catalytic isomerization of 2-ethyl-1-butene to 3-methyl 2-pentene (upper traces) as well as loss of 1-hexene (lower traces) in accordance with aspects of the present disclosure;

FIG. 6 graphically illustrates additional results of a catalytic isomerization of 2-ethyl-1-butene to 3-methyl 2-pentene (upper traces) as well as loss of 1-hexene (lower traces) in accordance with aspects of the present disclosure;

FIG. 7 graphically illustrates further results of a catalytic isomerization of 2-ethyl-1-butene to 3-methyl 2-pentene (upper traces) as well as loss of 1-hexene (lower traces) at time points prior to and following catalyst regeneration in accordance with aspects of the present disclosure; and

FIG. 8 graphically illustrates results of a catalytic isomerization of 2-ethyl-1-butene to 3-methyl 2-pentene (upper traces) as well as loss of 1-hexene (lower traces) in accordance with aspects of the present disclosure.

### DETAILED DESCRIPTION

Some implementations of the present disclosure will now be described more fully hereinafter with reference to the accompanying figures, in which some, but not all implementations of the disclosure are shown. Indeed, various implementations of the disclosure may be embodied in many different forms and should not be construed as limited to the implementations set forth herein; rather, these example implementations are provided so that this disclosure will be thorough and complete, and will fully convey the scope of the disclosure to those skilled in the art. Like reference numerals refer to like elements throughout. As used in the specification, and in the appended claims, the singular forms “a”, “an”, “the”, include plural referents unless the context clearly dictates otherwise.

Unless specified otherwise or clear from context, references to first, second or the like should not be construed to imply a particular order. A feature described as being above another feature (unless specified otherwise or clear from context) may instead be below, and vice versa; and similarly, features described as being to the left of another feature else may instead be to the right, and vice versa. Also, while reference may be made herein to quantitative measures, values, geometric relationships or the like, unless otherwise stated, any one or more if not all of these may be absolute or approximate to account for acceptable variations that may occur, such as those due to engineering tolerances or the like.

All ranges disclosed herein are inclusive of the endpoints, and the endpoints are independently combinable with each other (e.g., ranges of “up to 25 wt. %, or, more specifically, 5 wt. % to 20 wt. %”, is inclusive of the endpoints and all intermediate values of the ranges of “5 wt. % to 25 wt. %,” etc.). “Combination” is inclusive of blends, mixtures, alloys, reaction products, and the like.

As used herein, unless specified otherwise or clear from context, the “or” of a set of operands is the “inclusive or” and thereby true if and only if one or more of the operands is true, as opposed to the “exclusive or” which is false when all of the operands are true. Thus, for example, “[A] or [B]” is true if [A] is true, or if [B] is true, or if both [A] and [B] are true.

### Linear Alpha-Olefin (LAO) Purification Process and Catalyst System

Linear alpha-olefins (LAOs) are olefins represented by the general chemical formula CₓH₂ₓ and include 1-butene, 1-hexene, 1-octene, 1-decene, 1-dodecene, 1-tetradecene, 1-hexadecene, 1-octadecene, higher blends of C₂₀-C₂₄, C₂₄-C₃₀, and C₂₀-C₃₀ olefins. LAOs may be distinguished from additional mono-olefins with similar molecular formulae, for example, by the linearity of their hydrocarbon chains as well as the position of the double bond at the primary (or “alpha”) position. Linear alpha-olefins encompass a commercially valuable class of compounds, and are utilized as intermediates for the production of detergents, synthetic lubricants, copolymers, coatings, plasticizers, and additional products.

Existing processes for the production of linear alpha olefins typically rely on the oligomerization of ethylene. For example, linear alpha olefins can be prepared by the catalytic oligomerization of ethylene in the presence of various catalyst systems. Such reactions may be carried out in any reactor, such as a loop reactor, a plug-flow reactor, or a bubble column reactor. Oligomerization can occur at temperatures of 10 to 200° C., for example, 20 to 100° C., for example, 50 to 90° C., for example, 55 to 80° C., for example, 60 to 70° C. Operating pressures can be 1 to 5 MegaPascals (MPa), for example, 2 to 4 MPa. The process can be continuous and mean residence times can be 10 minutes to 20 hours, for example 30 minutes to 4 hours, for example, 1 to 2 hours. Residence times can be chosen so as to achieve the desired conversion at high selectivity. The resulting product stream containing linear alpha olefins is then subjected to a purification process.

Many commercially available processes for the enhancement of desirable species comprised in a linear alpha-olefin (LAO) stream are based on distillation. For example, the enrichment of LAOs such as 1-hexene can be performed by commercial distillation processes capable of removing impurities characterized by appreciably different (lower or higher) boiling points than 1-hexene (with a boiling point of about 63-64° C.) such as extractive distillation or azeotropic distillation. Distillation can occur, for example, at temperatures in a range of about 30° C. to about 80° C., for example, about 35° C. to about 70° C., for example, about 40° C. to about 65° C., for example, about 45° C. to about 60° C. Operating pressures can be in a range of about 0.5 atmospheres (atm) to 5 atmospheres (atm), for example, about 1 atm to about 2 atm.

The oligomerization process can be conducted in solution using an inert solvent, which is advantageously non-reactive with the catalyst composition. Examples of desirable organic solvents can include, but are not limited to, aromatic hydrocarbon solvents which can be unsubstituted or substituted with halogens, for example, toluene, benzene, xylene, monochlorobenzene, dichlorobenzene, chlorotoluene; aliphatic paraffin hydrocarbons, for example, pentane, hexane, heptane, octane, nonane, decane; alicyclic hydrocarbon compounds, for example, cyclohexane, decahydronaphthalene; and halogenated alkanes, for example, dichloroethane and dichlorobutane.

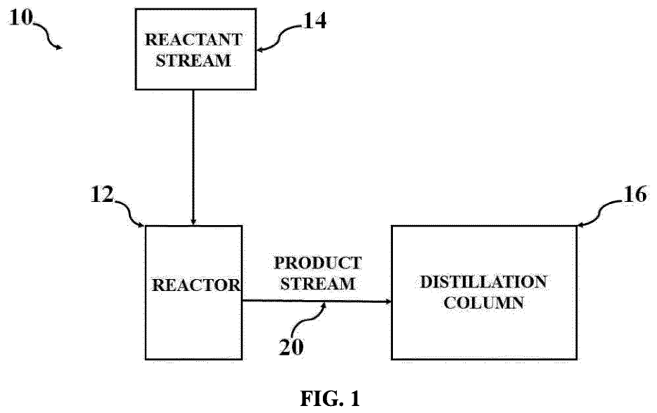

According to the present disclosure, a catalyst is positioned to receive a LAO product stream, the catalyst capable of isomerizing an alkyl-butene fraction into an alkyl-pentene fraction to enhance separation of one or more impurities in the LAO product stream. As illustrated in FIG. 1, the system 10 can include a reactor 12, a linear alpha-olefin (LAO) reactant stream 14, and a distillation column 16. In normal production mode, reactants 14, such as an LAO reactant stream comprising 1-hexene and impurities such as alkyl-butenes, including 2-ethyl-1-butene, can be fed into the reactor 12 comprising a catalyst system, such as a zeolite catalyst system, such as a zeolite catalyst system capable of regeneration, typically in the form of a packed bed of catalyst material. Typical operation conditions for the catalyst bed will include a pressure of about 5 psig or less, a temperature in a range of about 40° C. to about 60° C., and a liquid hourly space velocity (LHSV) in a range of about 0.5 h⁻¹ to about 10 h⁻¹.

The zeolite catalyst system of reactor 12 can catalytically isomerize impurities such as 2-ethyl-1-butene into isomers that are more easily separable from the resulting LAO product stream. For example, the zeolite catalyst may be optimally configured for isomerizing 2-ethyl-1-butene into either (cis- or trans-) isomer of 3-methyl-2-pentene, both of which are characterized by significantly higher boiling points (about 67-68° C.) than 2-ethyl-1-butene, with a boiling point (about 63.4° C.) similar to 1-hexene (about 63.85° C.).

The number of reactors 12 (which, in certain aspects, may interchangeably be referred to herein as catalyst beds) that are operable within the system 10 may vary. For instance, the system may have from one to five reactors, including a system having two reactors. Two or more reactors in system 10 may, in certain embodiments, be in direct fluid communication with each other. Alternatively, each of the two or more reactors of system 10 may operate as standalone reactors when, for example, at least one of the reactors is offline, is being serviced or is in need of service, etc., and each of the two or more reactors are configured to regenerate the zeolite catalyst therein under an environment substantially free of oxygen such as under an inert gas stream. Substantially free of oxygen may be a stream with 100 ppm or less, 50 ppm or less, 20 ppm or less, 10 ppm or less, 5 ppm or less, or 1 ppm or less oxygen. In related embodiments, two or more reactors may be positioned (in series or in parallel) and fluidly integrated with at least one distillation column 16.

After the reaction, a treated LAO product stream 20 can be directed into one or more distillation columns 16, wherein the treated LAO product stream can include the produced linear alpha olefins, such as C₄-C₂₀₊ olefins including 1-hexene. The one or more distillation columns 16 can be configured to separate one or more linear alpha-olefins in the LAO product stream by increasing or decreasing the operating temperature based on the boiling point of the desired linear alpha-olefin(s). For instance, distillative separation of 1-hexene may be achieved by operating distillation column 16 at approximately the boiling point of 1-hexene (about 63.85° C.).

Following the processing of one or more LAO reactant streams, the zeolite catalyst may become saturated and/or deactivated. Regeneration of the zeolite catalyst may be achieved, in certain aspects, by treating the catalyst under appropriate conditions for regeneration. For instance, regeneration of the zeolite catalyst may comprises treating the zeolite catalyst in a environment substantially free of oxygen such as in the presence of a continuous inert gas stream, for example, in nitrogen (N₂) or argon (Ar) stream, at a temperature in a range of about 150° C. to about 400° C. for example about 250° C. to about 300° C.

In certain aspects, the light alpha-olefin (LAO) reactant stream may be processed and/or manipulated for maximizing the concentration and weight percentage of 1-hexene in the stream. In this regard, 1-hexene is characterized by a boiling point of about 63-64° C., while a prevalent isomer of 2-ethyl-1-butene, a common impurity in LAO streams, has a similar boiling point of about 64.67° C. However, 2-ethyl-1-butene can be converted to a second, higher boiling point isomer such as cis- or trans-3-methyl-2-pentene using an isomerization catalyst. This equilibrium limited reaction may be illustrated as shown below:

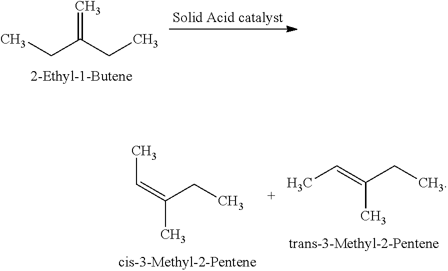

Isomerization catalysts adapted for use in the system of FIG. 1 included zeolite materials. In particular, the isomerization catalyst used in the present disclosure will advantageously include a combination of zeolite materials. The combination can include a first zeolite that is metal ion-exchanged and a second zeolite in H⁺ form. Reference to a metal ion-exchanged zeolite encompasses a zeolite wherein at least a portion of its ion exchange sites include a metal ion, such as the promoter metals noted below. Reference to a zeolite in H⁺ form encompasses a zeolite wherein at least a portion of its ion exchange sites contain hydrogen. One example zeolite combination is Na⁺ form zeolite Y (NaY) combined with H⁺ form zeolite Y (HY).

Without being bound by a theory of operation, it is believed that combining a metal ion-exchanged zeolite with an H⁺ form zeolite, particularly where both are large pore zeolites, provides a catalyst characterized by high 2-ethyl-1-butene (2E1B) isomerization, minimal undesirable linear alpha olefin conversion, and stable performance over time. By pairing a metal ion-exchanged zeolite with an H⁺ form zeolite, the acidity level of the catalyst can be balanced, delivering strong performance and good catalyst stability. It is noted that the H⁺ form zeolite can also include some metal ion-exchanged sites without departing from the invention.

The relative amount of each zeolite in the combination can vary, with a typical weight ratio of metal ion-exchanged zeolite to H⁺ form zeolite being in the range of about 5:1 to about 15:1, such as about 7:1 to about 12:1 or about 8:1 to about 10:1. In some embodiments, this weight ratio can be about 5:1 or more, or about 6:1 or more, or about 7:1 or more, or about 8:1 or more.

The phrase “zeolite” as used herein refers to framework aluminosilicates comprising or consisting of an extended array of three-dimensional network of SiO₄ and AlO₄ linked tetrahedra, which may be used, e.g., in particulate form, in combination with one or more promoter metals, as catalysts. Zeolites exhibit an extensive network having a substantially uniform pore distribution, with the average pore size typically being no larger than 20 Å. The pore sizes are defined by the framework ring size. According to one or more embodiments, it will be appreciated that defining zeolites by their structure type is intended to include both zeolites having that structure type and any and all isotypic framework materials such as SAPO, AlPO and MeAPO materials having the same structure type.

In more specific embodiments, reference to an aluminosilicate zeolite structure type limits the material to zeolites that do not purposely include phosphorus or other metals substituted in the framework. To be clear, as used herein, “aluminosilicate zeolite” excludes aluminophosphate materials such as SAPO, AlPO, and MeAPO materials, and the broader term “zeolite” is intended to include aluminosilicates and aluminophosphates. Aluminosilicate zeolites are crystalline materials, understood to have open 3-dimensional framework structures composed of corner-sharing TO₄ tetrahedra, where T is Al or Si.

In some embodiments, each zeolite is a large pore zeolite having an average pore size of greater than about 6 Å (e.g., zeolite X, zeolite Y, or beta zeolite). Example large pore zeolites have a framework type selected from the group consisting of AFI, AFR, AFS, AFY, ASV, ATO, ATS, BEA, BEC, BOG, BPH, BSV, CAN, CON, CZP. DFO, EMT, EON, EZT, FAU, GME, GON, IFR, ISV, ITG, IWR, IWS, IWV. IWW, JSR. LTF, LTL, MAZ, MEI, MOR, MOZ, MSE, MTW, NPO, OFF, OKO, OSI, RON, RWY, SAF, SAO, SBE, SBS, SBT, SEW, SFE, SFO, SFS, SFV. SOF, SOS, STO, SSF, SSY, USI, UWY, VET and mixtures or intergrowths thereof. For instance, the large pore zeolites in some embodiments are selected from the group consisting of framework types AFI, BEA, FAU, MAZ, MOR and OFF.

Zeolites generally comprise silica to alumina (SAR) molar ratios of 1 or greater. Zeolites for use in the disclosed catalyst compositions are not particularly limited in terms of SAR values, although the particular SAR value associated with a zeolite may, in some embodiments, affect the performance of the catalyst composition into which it is incorporated (e.g., particularly after aging). The molar ratio of silica-to-alumina (“SAR”) of the zeolites used in the present disclosure can vary over a wide range, but is generally 2 or greater. For instance, the first or second zeolite may have a SAR of from about 1 to about 1000 or about 2 to about 100, or about 3 to about 50 (e.g., ranges of about 2 to about 30 or about 3 to about 20 or about 4 to about 15).

The first zeolite and the second zeolite can be, for example, the same type of zeolite framework with the same silica-to-alumina ratio, the same type of zeolite framework with different silica-to-alumina ratio, or different zeolite frameworks. Cations that balance the charge of the anionic framework are loosely associated with the framework oxygen of a zeolite, and the remaining pore volume may be potentially filled with water molecules. The non-framework cations are generally exchangeable, and the water molecules removable.

In certain embodiments, zeolites having the FAU crystalline structure are used, which are formed by 12-membered ring structures and have channels of about 7.4 Å. Examples of such zeolites include faujasite, zeolite X, zeolite Y, LZ-210, and SAPO-37. Such zeolites are characterized by a 3-dimensional pore structure with pores running perpendicular to each other in the x, y, and z planes, with secondary building units 4, 6, and 6-6. An example SAR range for the bulk FAU zeolite material is about 3 to about 6, typically with a unit cell size range of 24.35 to 24.65, as determined by X-ray diffraction analysis (XRD). Zeolite Y is particularly useful for certain embodiments of the invention. In certain embodiments, one of the selected FAU zeolites is used in alkali metal form, such as the Na⁺ form.

As is known in the art, zeolites can be metal ion-exchanged or metal-promoted, which refers to a zeolite comprising one or more metals that are intentionally added, as opposed to comprising impurities that may be inherent in the zeolite. Thus, for example, a metal promoter is a component that is intentionally added to enhance the activity of a catalyst, compared to a catalyst that does not have promoter intentionally added. Promoter metals can generally be selected from the group consisting of alkali metals and alkaline earth metals.

Zeolites comprising a promoter metal, such as an alkali metal, are commercially available, such as from Zeolyst International, Inc. (PA, USA), Clariant AG (Muttenz, CH), KMI Zeolite, Inc. (Pahrump, NV, USA), and W.R. Grace & Co. (Columbia, MD, USA). The alkali metal content of zeolites typically results from the zeolite formation process, which can comprise, for example, mixing a reaction mixture comprising water, an aluminum source, a silicon source (which can be an alkali metal silicate), and a structure-directing agent to form an aluminosilicate-containing solution, referred to as a “synthesis gel” or “gel”, and subjecting the synthesis gel to a crystallization process to crystallize the zeolite. Zeolites in H⁺ form are commercially available, such as from Fisher Scientific (Waltham, MA, USA), Alfa Aesar (Tewksbury, MA, USA), and Sigma-Aldrich Inc. (St. Louis, MO, USA). Alternatively, H⁺ form zeolites can be formed by ion exchange processes known in the art, such as by ion-exchange with NH₄⁺ followed by calcination to achieve the H⁺ form.

Catalyst compositions comprising multiple zeolites can be formed by mixing multiple zeolite materials with a binder, followed by extrusion and milling to desired size. Suitable binders include metal oxides, such as silica, alumina, titania, zirconia, ceria, or a combination thereof. Alumina binders include aluminum oxides, aluminum hydroxides, and aluminum oxyhydroxides. Aluminum salts and colloidal forms of alumina many also be used. Silica binders include various forms of SiO₂, including silicates and colloidal silica. The loading of the binder is typically about 1 wt. % to about 50 wt. %, based on the total weight of the catalyst composition. As an example, a binder may comprise an alumina of BET (N₂ adsorption) surface area of between 200-400 m²/g, and an average crystallite size in a range of about 1 micrometer (μm) to about 10 micrometers (μm), for example about 1.5 μm to about 3.5 μm, and comprise an average pore size in a range of about 3 nanometers (nm) to about 10 nanometers (nm), for example about 5 nm to about 8 nm.

The acidity of the catalyst composition of the present disclosure, such as the combination of the first zeolite with the second zeolite, can be expressed in several ways. For example, acidity can be calculated based on chemical formula or unit cell (uc) composition of the zeolite materials. In this manner, one can calculate Brönsted Acid Site (BAS) equivalent. As an example, BAS of HZSM-5 (see CBV5524 in Table 2 below) was calculated as follows: chemical formula of the zeolite H_{3.69}Al_{3.69}Si_{92.31}O₁₉₂ containing 3.69 H⁺ (or BAS) mol equivalent per unit cell or per 5769.89 g (MW of uc) of zeolite powder, that is, 0.64 mmol H⁺/g zeolite. In certain embodiments, the Brönsted Acid Site (BAS) equivalent is about 1.0 mmol/g catalyst or less, such as about 0.9 mmol/g catalyst or less or about 0.5 mmol/g catalyst or less, with example ranges including about 0.1 mmol/g catalyst to about 1.0 mmol/g catalyst or about 0.2 to about 0.9 or about 0.3 to about 0.7.

As noted above, the Brönsted acid site (BAS) of a catalyst can be calculated based on chemical formula of the zeolite content of the zeolite. Whereas total acid site, BAS (contribution from zeolite) plus Lewis acid site (contribution from both zeolite and alumina binder), can be characterized by the temperature-programmed desorption (TPD) of NH3. For example, the isomerization catalyst may have an ammonia desorption profile with less than about 2.0 mmol NH₃ desorbed per g catalyst with NH₃-TPD peak(s) maxima at less than about 500° C., for example, or less than about 1.5 mmol NH₃ desorbed per g catalyst or less than about 1.0 mmol NH₃ desorbed per g catalyst (e.g., about 0.5 to about 2.0 mmol NH3 desorbed per g catalyst. A test protocol for TPD of NH₃ is set forth in the example section.

In certain embodiments, the LAO product stream to be treated according to the present disclosure can be characterized by 1-hexene content and 2-ethyl-1-butene (2E1B) content. An example product stream comprises 1-hexene in an amount of 90 wt. % or greater (e.g., 95 wt. % or greater or 97% or greater), such as 90 wt. % to about 98 wt. %. An example product stream comprises a 2E1B concentration of about 0.7 wt. % or higher (e.g., about 0.8 wt. % or higher or about 0.9 wt. % or higher), such as about 0.7 wt. % to about 2.0 wt. %.

In certain aspects, the isomerization catalyst may be characterized based on how much of the undesirable first isomer is converted to the second isomer. For example, in certain embodiments, the isomerization catalyst is capable of converting about 90% by weight or greater of 2-ethyl-1-butene (e.g., about 95% or greater or about 97% or greater or about 99% or greater), such as about 95% to about 99.5%, to the second isomer. In some embodiments, the treated LAO product stream is characterized by a 2E1B concentration of equal to or less than about 0.3 wt. % or less than about 0.2 wt. % or less than about 0.1 wt. % (e.g., about 0.05 wt. % to about 0.3 wt. %).

The isomerization catalyst also may be characterized based on how much 1-hexene is undesirably converted to a different isomer, with example ranges including about 1.0% by weight or less of 1-hexene (e.g., about 0.75% or less or about 0.5% or less), such as about 0.1% to about 1.0% or about 0.1% to about 0.75% or about 0.1% to about 0.5%.

This technology is broadly applicable to isomerization reactions capable of enriching the concentration of commercially valuable compounds such as C₆ compounds as described herein, but also applicable for other reactions which are equilibrium limited. For example, such other reactions include isomerization reactions of other linear alpha-olefins, as well as metathesis reactions of C₂ to C₁₂ olefins, etherification reactions of C₄ and Cs olefins with alcohols (e.g., methanol, ethanol, or isoamyl alcohol) to form gasoline additives which increase octane number of the fuel, esterification, acetalization, and related processes.

### EXPERIMENTAL

### Example 1

An analysis of typical linear alpha-olefin (LAO) composition stream, with particular regard to 1-hexene (1H) isomeric composition stream, was compiled as shown in Table 1 and assessed on the basis of boiling points and compositional stream concentrations of the 1-hexene isomers present. 2-ethyl-1-butene (2E1B) was initially targeted, at least in part, due to its significance as the close boiling points (b.p.) of 1-hexene (b.p. 63.85° C.) versus isomer 2-ethyl-1-butene (b.p. 63.4° C.).

TABLE 1

| | | Concentration of 1- |
|---|---|---|
| LAO Component | Boiling Point (° C.) | Hexene Isomers (wt. %) |
| 2-ethyl-1-butene | 63.4 | 0.763 |
| 3-methyl-1-pentene | 54.0 | 0.00216 |
| N-hexane | 68.73 | 0.133 |
| Trans-3-hexene | 67 | 0.049 |
| Cis-3-hexene | 67 | 0.017 |
| Trans-2-hexene | 67.9 | 0.261 |
| Cis-2-hexene | 67.9 | 0.347 |
| Trans 3-methyl 2- | 67-68 | 0.00438 |
| pentene | | |
| Cis 3-methyl 2- | 67-68 | 0.00354 |
| pentene | | |
| 1-hexene | 63.85 | — |

A series of lab reactor tests were conducted with various isomerization catalysts to determine feasibility of using such catalysts to conveli 2-ethyl-1-butene (2E1B) to a different isomer, such as cis- or trans-3-methyl-2-pentene. Conversion percentages (wt. %) were determined for both 2E1B and 1H at a temperature of about 45° C., a pressure of about 1 atm, and at the liquid hourly space velocities (LHSVs) described in Table 2. The tested catalysts were used in the form of extrudates comprising 80 wt % zeolite and 20 wt % alumina binder.

The tested catalysts were as follows: extrudate of sodium zeolite Y (NaY) with SAR of 5.1 (Example 1; also referred to as CBV100); extrudate of the H⁺ form of zeolite Y (HY) with a SAR of 5.1 (Example 2; also referred to as CBV400); extrudate of a steam or acid stabilized H⁺ form of zeolite Y (stabilized HY) with a SAR of 30 (Example 3; also referred to as CBV720); extrudate of a steam or acid stabilized W form of zeolite Y (stabilized HY) with a SAR of 60 (Example 4; also referred to as CBV760); extrudate of a 9:1 weight ratio mixture of sodium zeolite Y (NaY) with SAR of 5.1 and H⁺ form of zeolite Y (HY) with a SAR of 5.1 (Example 5); extrudate of the W form of Zeolite Socony Mobil-5 (HZSM-5) with a SAR of 50 (Example 6; also referred to as CBV5524); extrudate of the H⁺ form of Zeolite Socony Mobil-5 (HZSM-5) with a SAR of 280 (Example 7; also referred to as CBV28014); and AMBERLYST®15 (Example 8). Noted that the catalysts named CBV100, CBV400, CBV720, CBV760, CBV5524, and CBV28014 were obtained from Zeolyst International, Inc.

TABLE 2A
(Powders)

| | | | | BAS, |
|---|---|---|---|---|
| | | | Powder Zeolite Unit Cell | mmole/g- |
| | Catalyst | | Composition | zeolite¹ |
| Example | Zeolite | SAR² | | Powder |
| | Powder | | | |
| | or | | | |
| | Extrudate | | | |
| | CBV100, | 5.1 | Na_{54.1}A1₅₄.1Si_{137.9}O₃₈₄ | 0 |
| | NaY | | | |
| | CBV400, | 5.1 | Na_{0.1}H_{54.0}A1_{54.1}Si_{137.9}O₃₈₄ | 4.68 |
| | HY⁴ | | | |
| | CBV720 | 30.0 | H₁₂A1₁₂Si₁₈₀O₃₈₄ | 1.04 |
| | (Stabilized | | | |
| | HY)⁵ | | | |
| Example | Zeolite | SAR² | | Powder |
| | Powder | | | |
| | or | | | |
| | Extrudate | | | |
| | CBV760 | 60.0 | H_{6.19}A1_{6.19}Si_{185.81}O₃₈₄ | 0.54 |
| | (Stabilized | | | |
| | HY)⁵ | | | |
| | CBV5524 | 50 | H_{3.69}A1.₃₆₉Si_{92.31}O₁₉₂ | 0.64 |
| | HZSM-5 | | | |
| | CBV28014 | 280 | H_{0.68}A1_{0.68}Si_{95.32}O₁₉₂ | 0.12 |
| | HZSM-5 | | | |

TABLE 2B
(Extrudates)

| | Catalyst | | BAS, mmole/g- | Activity, % Conversion at 3-9 LHSV | | | | |
|---|---|---|---|---|---|---|---|---|
| | Zeolite Powder | | zeolite¹ | | | | | Catalyst Selection |
| Example | or Extrudate | SAR² | Extr.³ | LHSV | TOS, h | 2E1B | 1-H | Observations |
| 1 | CBV100, | 5.1 | 0 | 3.0 | 3.0 | 100 | 0.1 | Initial 1H conversion was |
| | Extrudate | | | | | | | low; 2E1B conversion |
| | | | | | | | | declined over time |
| 2 | CBV400 | 5.1 | 3.74 | 5.0 | 3.2 | 97.9 | 4.2 | Initial 1H conversion was |
| | Extrudate | | | | | | | high; overall catalyst |
| | | | | | | | | performance was not |
| | | | | | | | | acceptable |
| 3 | CBV720, | 30 | 0.83 | 8.6 | 3.1 | 99.7 | 11.1 | Initial 1H conversion was |
| | Extrudate | | | | | | | very high; overall catalyst |
| | | | | | | | | performance was not |
| | | | | | | | | acceptable |
| 4 | CBV760, | 60 | 0.43 | 8.6 | 2.9 | 97.9 | 19.5 | Initial 1H conversion was |
| | Extrudate | | | | | | | very high; overall catalyst |
| | | | | | | | | performance was not |
| | | | | | | | | acceptable |
| 5 | CBV100/ | | 0.37 | 3.6 | 3.4 | 98.7 | 0.4 | Initial 1H conversion was |
| | CBV400 | | | | | | | low; catalyst was found |
| | (9:1)⁶ | | | | | | | to be stable |
| 6 | CBV5524 | 50 | 0.51 | 8.6 | 2.9 | 97.4 | 2.5 | Initial 1H conversion was |
| | Extrudate | | | | | | | high; fast deactivation |
| | | | | | | | | was observed |
| 7 | CBV28014 | 280 | 0.10 | 5.1 | 2.9 | 96.7 | 1.3 | Initial 1H conversion was |
| | Extrudate | | | | | | | high; fast deactivation |
| | | | | | | | | was observed |
| 8 | Amberlyst-15 | | | 8.6 | 2.9 | 97.6 | 1.9 | Initial 1H conversion was |
| | | | | | | | | high |

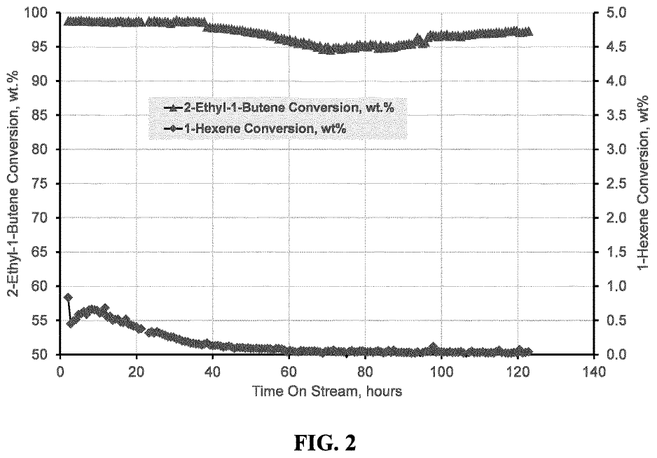

The zeolite unit cell composition, acid site, and catalytic test results are shown in Table 2. As described in Table 2, many of tested catalysts were deleteriously characterized by high 1H conversion rates and/or rapid catalyst deactivation. However, Example 5, which comprised a mixture of CBV100/CBV400 (NaY/HY), at a 9:1 weight ratio with respect to NaY:HY, was beneficially found to exhibit both high 2E1B conversion and low 1H conversion (as shown in FIG. 2) and sufficient catalyst stability. Examples 6 and 7, which utilized the medium pore ZSM-5 zeolite, exhibited unacceptably high 1H conversion and catalyst performance deteriorated quickly.

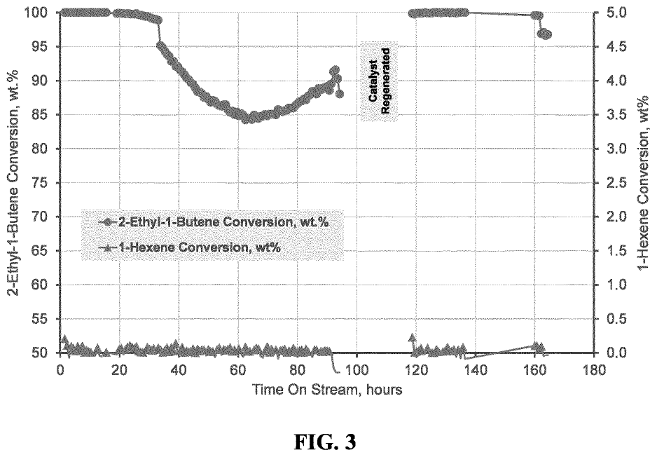

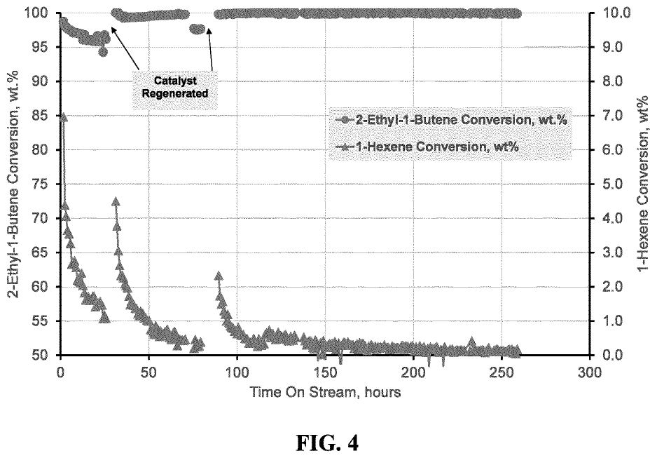

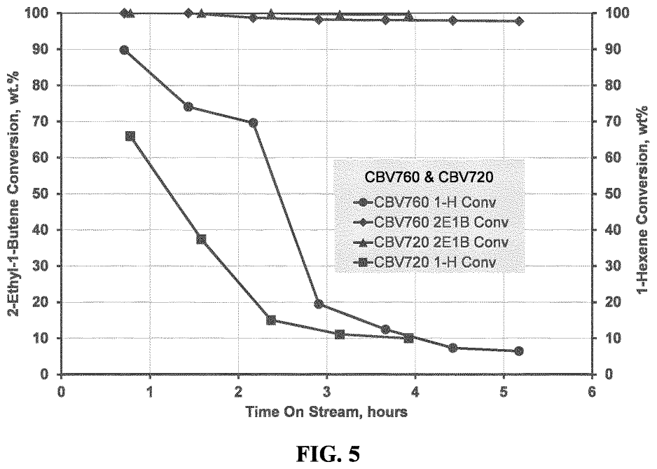

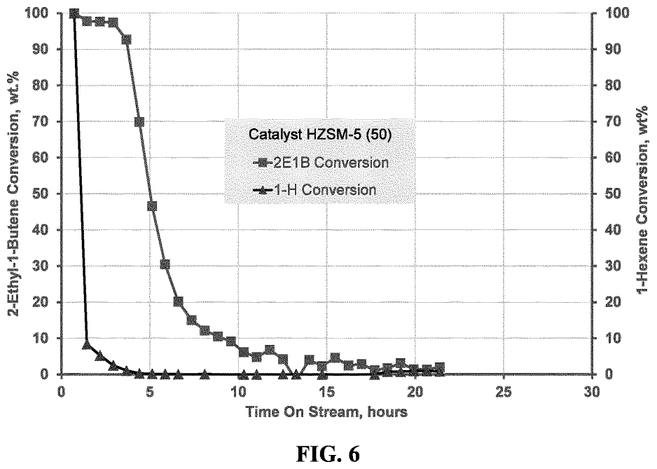

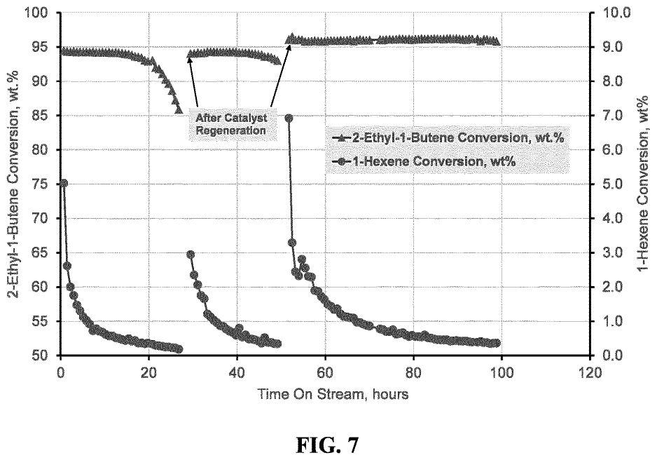

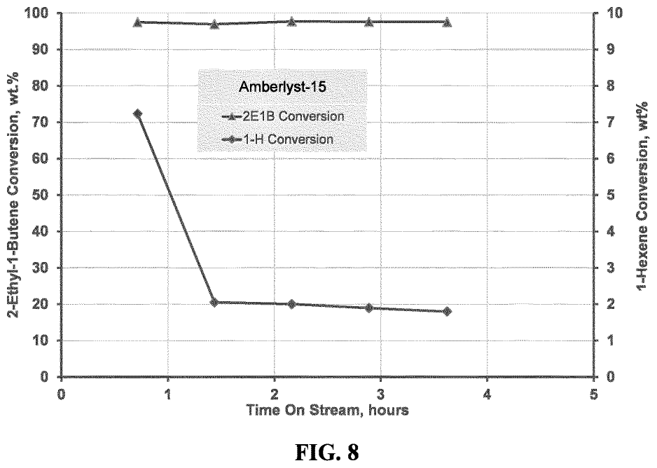

Additional 2E1B and 1H catalytic conversion profiles are further provided in the accompanying figures for NaYCBV100 alone (FIG. 3), HY/CBV400 alone (FIG. 4), stabilized HY with SAR of 30.0 (CBV720) and 60.0 (CBV760) (FIG. 5), HZSM-5 (50)/CBV5524 (FIG. 6), HZSM-5 (280)/CBV28014 (FIG. 7), and AMBERLYST® 15 (FIG. 8).

### Example 2

The testing of Example 1 was repeated for several of the catalyst samples and the data is provided in Table 3 below. Acidity of some of the tested materials was also quantified using temperature programmed desorption (TPD) of NH₃. In present disclosure, the total acidity of the catalyst was determined by NH₃-TPD analysis using conventional TCD-TPD equipment (AutoChem II 2920; Micromeritics Instrument Corp., Norcross, GA, USA). A weighted amount (typically 200 mg) of catalyst was preactivated by drying under He flow (30 cm³/min) at max temperature of 550° C., and after cooling down to 100° C., NH₃ was adsorbed on catalyst by flowing 5% NH₃ in He, rate 30 cm³/min at 100° C. for 30 min. Physical adsorbed NH₃ was then removed by flowing He (50 cm³/min) at 100 for 1 h. Ammonia was then desorbed by heating the catalyst sample from 100° C. up to 550° C. at a rate of 10° C./min and under a helium flow rate of 30 cm³/min. TCD signal attributed to desorbed NH₃ was measured as a function of time and temperature. Acid site was determined from NH₃ desorbed amount under the peak area.

TABLE 3

| | | | | | Acidity, mmol/ g- | | | |
|---|---|---|---|---|---|---|---|---|
| | | | | | catalyst | | | |
| Catalyst | | | | | NH₃ | NH₃ | | |
| | Zeolite | | | | de- | de- | Activity, % Conversion at | |
| Zeolite Powder | Extruded | Catalyst Composition,² wt. % | | | sorbed <300° | sorbed <550° | given LHSV^{a} @3 h | |
| (SAR)¹ | Catalyst² | Na | Si | Al | C. | C. | 2E1B | 1-H |
| NaY (5.1) | NaY (5.1) | 6.9 | 23.9 | 14.2 | 0.83 | | 100 (3.0) | 0.1 (3.0) |
| NaHY (5.1) | NaHY (5.1) | 1.29 | 26.09 | 19.5 | (see note)^{b} | 097^{b} | 97.9 (5.0) | 4.1 (5.0) |
| NaY (5.1)/ | | | | | | | 98.8 (3.6) | 0.5 (3.6) |
| NaHY (5.1)^{c} | | | | | | | | |
| ZSM-5 (50) | HZSM-5 | Not | 33.33 | 12.35 | 0.37 | 0.34 | 99.4 (8.6) | 2.6 (8.6) |
| | (50) | determined | | | | | | |
| ZSM-5 (280) | HZSM-5 | Not | 34.89 | 10.94 | 0.23 | 0.06 | 97.7 (5.1) | 1.2 (5.1) |
| | (280) | determined | | | | | | |

¹Number shown in parenthesis is SiO₂/Al₂O₃ Ratio (SAR) of powder zeolite
²Extruded catalyst, powder zeolite bound with 20 wt % alumina binder.
^{a}At LHSV/h shown in parenthesis
^{b}NH₃ desorbed showing broad peak between 100° C. and 500° C. with peak maximum at 200° C.
^{c}Physical mixture (9:1 ratio by weight)

The results are shown in Table 3. All of the tested catalysts were able to convert a large percentage of 2E1B to an isomer capable of being more easily removed from the resulting LAO product stream. However, the physical mixture of NaY (5.1)/NaHY (5.1) provided the strongest and most stable performance, consistent with Example 1.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention.

Many modifications and other implementations of the disclosure will come to mind to one skilled in the art to which this disclosure pertains having the benefit of the teachings presented in the foregoing descriptions and the associated figures. Therefore, it is to be understood that the disclosure is not to be limited to the specific implementations disclosed herein and that modifications and other implementations are intended to be included within the scope of the appended claims. Although specific terms are employed herein, they are used in a generic and descriptive sense only and not for purposes of limitation.

1. A catalyst composition comprising a mixture of a first zeolite and a second zeolite and an optional metal oxide binder, the first and second zeolites each having an average pore size of greater than about 6 Å, wherein the catalyst composition comprises a Brönsted Acid Site (BAS) equivalent of about 1.0 mmol/g catalyst or less, and wherein the first zeolite is metal ion-exchanged with one or more metals selected from the group consisting of alkali metals, alkaline earth metals, and combinations thereof and the second zeolite is in H⁺ form, and wherein the first and second zeolite are present in the mixture at a weight ratio of first zeolite to second zeolite in a range of about 5:1 to about 15:1.

2. The catalyst composition of claim 1, wherein the first zeolite is metal ion-exchanged with sodium.

3. The catalyst composition of claim 1, wherein each of the first and second zeolite has a 12-membered ring framework structure independently selected from the group consisting of AFI, AFR, AFS, AFY, ASV, ATO, ATS, BEA, BEC, BOG, BPH, BSV, CAN, CON, CZP, DFO, EMT, EON, EZT, FAU, GME, GON, IFR, ISV, ITG, IWR, IWS, IWV, IWW, JSR, LTF, LTL, MAZ, MEI, MOR, MOZ, MSE, MTW, NPO, OFF, OKO, OSI, RON, RWY, SAF, SAO, SBE, SBS, SBT, SEW, SFE, SFO, SFS, SFV, SOF, SOS, STO, SSF, SSY, USI, UWY, VET, and combinations thereof.

4. The catalyst composition of claim 1, wherein each of the first and second zeolite has a FAU or BEA framework structure, each of the first and second zeolite having a silica-to-alumina ratio (SAR) from about 1 to about 100.

5. The catalyst composition of claim 4, wherein each of the first and second zeolite is zeolite X, zeolite Y, or beta zeolite.

6. The catalyst composition of claim 1, wherein the first and second zeolite are present in a weight ratio of first zeolite to second zeolite in a range of about 7:1 to about 12:1.

7. The catalyst composition of claim 1, wherein the catalyst composition comprises a Brönsted Acid Site (BAS) equivalent of about 0.9 mmol/g catalyst or less.

8. The catalyst composition of claim 1, wherein the catalyst composition is in the form of an extrudate comprising a mixture of the first zeolite, the second zeolite, and a binder.

9. (canceled)

10. A method of isomerizing an alkyl-butene fraction into an alkyl-pentene fraction in a linear-alpha olefin (LAO) product stream comprising contacting the LAO product stream with a catalyst composition of claim 1.

11. A process for producing a purified linear-alpha olefin (LAO) product stream, the process comprising:
a) introducing a LAO product stream comprising an alkyl-butene fraction into a catalyst bed comprising a catalyst composition according to claim 1, which can be regenerated under an environment substantially free of oxygen, and wherein the catalyst composition is in fluid connection with the LAO product stream to catalytically isomerize the alkyl-butene fraction into an alkyl-pentene fraction and form a treated LAO product stream; and
b) directing the treated LAO product stream into at least one distillation column having a plurality of stacked stages to produce a purified LAO product stream.

12. The process of claim 11, wherein the alkyl-butene is 2-ethyl-1-butene (2E1B) and the alkyl-pentene is cis- or trans-3-methy-2-pentene.

13. The process of claim 12, wherein:
a. the LAO product stream has a 2E1B concentration of about 0.7 wt. % or higher and the treated LAO product stream is characterized by a 2E1B concentration of equal to or less than about 0.3 wt. %; and/or
b. treatment with the zeolite catalyst catalytically isomerizes about 90% by weight or more of the 2E1B and about 1.0% by weight or less of 1-hexene in the LAO product stream; and/or
c. the LAO product stream comprises 1-hexene in an amount of 90 wt. % or greater; and/or
d. the purified LAO product stream is characterized by a 1-hexene product purity of about 99.5 wt. % or greater.

14. The process of claim 11, wherein the introducing step occurs at one or more of the following conditions:
a. a liquid hourly space velocity (LHSV) of about 0.5 h⁻¹ to about 10 h⁻¹;
b. a pressure of about 5 psig or less; and
c. a temperature of about 40 to about 60° C.

15. The process of claim 11, wherein at least two catalyst beds comprising the zeolite catalyst are operated in parallel and in fluid connection with the LAO product stream such that at least one catalyst bed can be regenerated under an environment substantially free of oxygen while continuing to treat the LAO product stream, and wherein the process further comprises regenerating one of the catalyst beds by treating the catalyst bed with an inert gas stream at a temperature in a range of about 150° C. to about 400° C.

16. The catalyst composition of claim 5, wherein the first zeolite is Na⁺ form zeolite Y (NaY) and the second zeolite is H⁺ form zeolite Y (HY).

17. The catalyst composition of claim 7, wherein the catalyst composition comprises a Brönsted Acid Site (BAS) equivalent of about 0.5 mmol/g catalyst or less.
